(19) United States
(12) Patent Application Publication
(10) Pub. No.: US 2006/0183952 A1
(43) Pub. Date: Aug. 17, 2006

(54) Process for the removal of benzene from gasoline streams

(75) Inventors: Arvids Judzis, Houston, TX (US); Richard M. Foley, Cotopaxi, CO (US); Kenneth H. Johnson, Missouri City, TX (US)

Correspondence Address: KENNETH H. JOHNSON, P.O. BOX 630708, HOUSTON, TX, 77263, US

(73) Assignee: CATALYTIC DISTILLATION TECHNOLOGIES

(21) Appl. No.: 11/059,150

(22) Filed: Feb. 16, 2005

(51) Int. Cl. C07C 2/00

(52) U.S. Cl. 585/324

(57) ABSTRACT

A process for producing debenzenized and isomerized complex refinery streams, such as gasoline blending stock, by hydrogenating benzene, contained in a mixed hydrocarbon stream in which for example a reformate naphtha stream containing benzene is hydrogenated with a selective hydrogenation catalyst to convert benzene into cyclohexane and contacting the debenzenized stream containing cyclohexane with a selective isomerization catalyst to isomerize cyclohexane to methyl cyclopentane and incidentally at the same time isomerize normal paraffins to the corresponding isoparaffins.

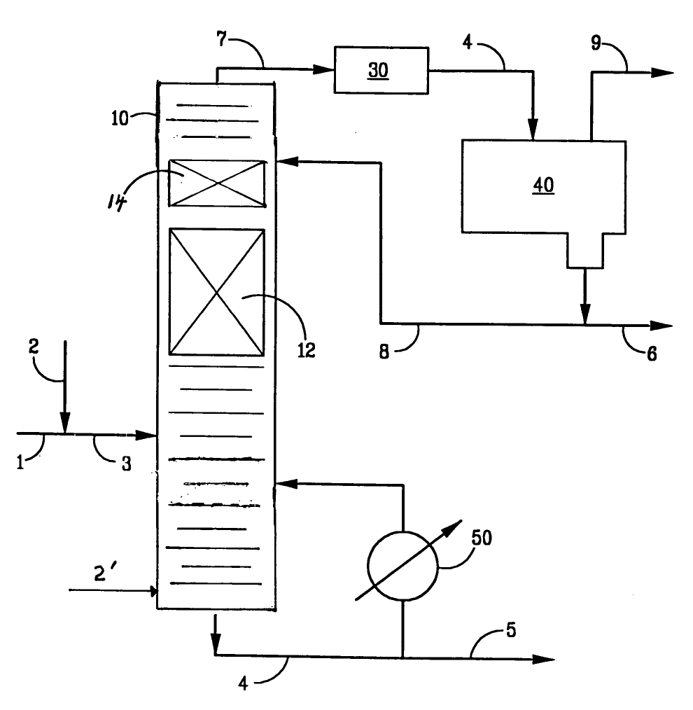

### BACKGROUND OF THE INVENTION

1. Field of the Invention

The present invention relates to the selective hydrogenation of benzene in a stream, such as naphtha from a cracking process or from a catalytic reformer, to make cyclohexane and reduce the benzene content and the isomerization of the cyclohexane and C₅-C₆ paraffins. More particularly the invention relates to a process wherein the hydrogenation of the benzene and isomerization of the cyclohexane and paraffins uses a catalytic distillation operating mode.

2. Related Information

The reduction in the lead content of gasolines and the ban on use of lead antiknock compounds have led to a search for other ways to improve the octane number of blending components for gasoline. The alternatives to uses of lead antiknock compounds are chemical processing and the use of other additives.

One common process long used by the refinery industry to upgrade raw naphtha to high octane gasoline is catalytic reforming. In catalytic reforming the raw naphtha having a boiling range of circa 115-350° F. is passed over an alumina supported noble metal catalyst at elevated temperatures (circa 92-1020° F.) and moderate pressure (circa 50-350 psig). The catalyst “reforms” the molecular structures of the hydrocarbons contained in the raw naphtha by removing hydrogen and rearranging the structure of the molecules so as to improve the octane number of the naphtha.

Because of the multiplicity of the compounds in the raw naphtha, the actual reactions which occur in catalytic reforming are numerous. Some of the many resulting products are aryl or aromatic compounds, all of which exhibit high octane numbers. The aryl compounds produced depend upon the starting materials which in a refinery are controlled by the boiling range of the naphtha used and the crude oil source. The “reformed” product from a catalytic reforming process is commonly called reformate and is often separated into two fractions by conventional distillations—a light reformate having a boiling range of circa 1 15-250° F. and a heavy reformate having a boiling range of circa 250-350° F. The aryl compounds in each fraction are thus dependent upon their boiling points. The lower boiling or lighter aryl compounds, e.g., benzene, toluene and xylenes, are contained in the light reformate and higher boiling aryl compounds are contained in the heavy reformate.

Both reformate fractions may be blended into gasoline. Until the EPA mandate requiring an elimination of most benzene from gasoline (general requirements for reformulated gasoline specify a maximum of 1.0 vol. % benzene) benzene was a solution to offset octane loss by the elimination of lead. Now benzene must be removed or converted to more benign components, while maintaining the octane of the gasoline. One effective means to achieve this is to alkylate the benzene, however the olefin streams for this purpose may be expensive or otherwise employed.

Hydrogenation of the benzene is an alternative for removing that aromatic compound from gasoline streams. One example of this process is disclosed by Hsieh, et al in U.S. Pat. No. 5,210,348 wherein hydrogenation of the benzene fraction is used alone or in combination with alkylation. Peterson in U.S. Pat. No. 2,373,501 discloses a liquid phase process for the hydrogenation of benzene to cyclohexane wherein a temperature differential is maintained between the top of the catalyst bed where benzene is fed and the outlet where substantially pure cyclohexane is withdrawn. The temperature differential is due to the change in the exothermic heat of reaction released as less and less benzene is converted as the concentration of benzene decreases. The benzene/cyclohexane flow is contacted by the hydrogen in countercurrent flow. Temperature control coils are disposed within the reactor to maintain the temperature differential if the exothermic heat of reaction is not sufficient or to cool the bed if too much heat is released. Peterson recognizes that although the bulk of his reaction takes place in the liquid phase a portion of the benzene and cyclohexane will be vaporized, especially near the top of the reactor where the benzene concentration is highest and conversion is highest. A reflux condenser is provided to condense the condensible material and return it to the reactor. Thus, a substantial portion of the heat of reaction is removed by condensation of the reactants vaporized throughout the reaction.

Larkin, et al. in U.S. Pat. No. 5,189,233 disclose another liquid phase process for the hydrogenation of benzene to cyclohexane. However, Larkin, et al utilize high pressure (2500 psig) to maintain the reactants in the liquid state. In addition Larkin, et al disclose the use of progressively more active catalyst as the concentration of benzene decreases to control the temperature and unwanted side reactions.

U.S. Pat. No. 5,830,345 discloses hydrogenating benzene and isomerizing the cyclohexane product in a catalytic distillation reactor using a dual function catalyst, which is not specific for either reaction. The very close boiling points of the benzene and cyclohexane made the less efficient dual function catalyst approach appear as an unavoidable solution.

The low octane cyclohexane is not a desirable gasoline blending component, and its isomer, methyl cyclopentane is a more desirable blending component. It is a feature of the present invention that the reduction of benzene by hydrogenation to cyclohexane and/or the isomerization of the cyclohexane may be implemented with a catalytic distillation mode. Another advantage of the present invention is the use of selective hydrogenation catalyst for the hydrogenation of benzene and the use of selective isomerization catalyst for the isomerization, thereby obtaining the maximum benefit from each of the two separate and distinct process steps compared to a dual function catalyst which is not specifically designed for either.

### SUMMARY OF THE INVENTION

The present invention comprises a process for debenzening complex refinery streams such as gasoline blending stock, e.g., a reformate naphtha stream by hydrogenating the benzene to cyclohexane benzene hydrogenation product, and improving the octane number of the resultant product by isomerizing cyclohexane to methyl cyclopentane which has a higher octane number than cyclohexane.

In one embodiment the process comprises: (a) feeding a complex refinery stream, containing benzene and normal C₅-C₆ paraffins, to a distillation column reactor into a feed zone and hydrogen into said feed zone; and (b) concurrently: (1) contacting a benzene rich portion of said complex refinery stream, preferably C₅-C₆ naphtha, and said hydrogen with a fixed bed selective hydrogenation catalytic distillation structure in a distillation reaction zone under hydrogenation conditions; and (2) fractionating said reaction mixture: (i) to bring said benzene into the distillation reaction zone to selectively react at least a portion thereof with said hydrogen to form a reaction mixture containing cyclohexane and unreacted hydrogen and (ii) to fractionate said cyclohexane and C₅-C₆ paraffins from said reaction mixture; and (3) contacting said cyclohexane normal C₅-C₆ paraffins and hydrogen with a selective isomerization catalytic distillation structure in a distillation reaction zone to produce methyl cyclopentane and C₅-C₆ branched paraffins. Any unreacted benzene is also carried overhead.

In another embodiment the hydrogenation of benzene is carried out as described above and a sidedraw stream is taken immediately above the hydrogenation distillation reaction zone. The sidedraw is taken to an isomerization reaction operated in the catalytic distillation mode containing a catalyst selective for the isomerization of cyclohexane to methyl cyclopentane. The cyclohexane is isomerized by contacting the sidedraw stream with the selective hydrogenation catalyst under isomerization conditions whereby cyclohexane is isomerized to methyl cyclopentane with an overheads from the isomerization reaction being returned to a point above the sidedraw and a bottoms from the isomerization reaction being recovered, returned at a point below the hydrogenation distillation reaction zone and/or recycled to the feed.

In another embodiment a benzene containing complex refinery stream is fed to a distillation column splitter to recover a lighter fraction (a benzene rich portion of said complex refinery stream, preferably C₅-C₆ naphtha) as overheads and a heavier portion (C7+naphtha) as bottoms and treating the overheads in a downflow reactor. The C₅-C₆ paraffins in the light fraction also contact the isomerization catalyst and are isomerized to the isoparaffins.

By having the C₅-C₆ paraffins in the feed stream come into contact with the isomerization catalyst and also convert to the iso C₅-C₆ paraffins, a further beneficial increase in octane is obtained.

### BRIEF DESCRIPTION OF THE DRAWING

The FIGURE is a schematic flow diagram of the invention.

### DETAILED DESCRIPTION OF THE INVENTION

Any complex refinery streams containing a minor amount of benzene and which need to be and can be reduced in benzene content by hydrogenation, is appropriate for use in the present process. By “complex refinery streams”, it is intended to mean the normally liquid product streams found in a refinery from cokers, FCC units, reformers, hydrocrackers, hydrotreaters, delayed cokers, distillation columns, etc. which streams comprise a range of chemical constituents, mainly hydrocarbonaceous, and having a broad boiling point range. The preferred complex refinery stream is selected from the group consisting of reformate, light reformate, heart-cut reformate, FCC gasoline, FCC light gasoline, coker gasoline, and coker light gasoline. In accordance with some embodiments of the invention a light reformate is most preferred and comprises a complex aromatics-containing stream containing a minor amount of benzene, produced in a refinery reforming unit, and generally having a boiling point range of 60 to 220° F. In such instances the preferred benzene concentration of the light aromatics-containing streams is between about 1% and 40% by volume, more preferably between about 2% and 30% and most preferable between about 5% and 25%. In other embodiments of the invention a full boiling range reformate is the preferred feed. In such instances the reformate will generally have a boiling point range of 60 to 400° F. and the preferred benzene concentration of the full boiling range aromatics-containing stream is between about 1% and 20% by volume, more preferably between about 2% and 15% and most preferable between about 3% and 10%. The concentration of olefins in these olefin-containing streams may vary, but is preferably between about 5% to 40% olefin by volume, and more preferably between about 10% and 30% by volume.

A typical benzene containing naphtha is a light reformate having the following characteristics:

TABLE I
Typical Light Reformate Analysis

| Component | wt. % |
|---|---|
| C₃ to C₈ non aromatics | 55.5 |
| Benzene | 9.5 |
| Toluene | 32.5 |
| Ethylbenzene + Xylenes | 2.0 |
| C₉ aromatics | 0.2 |
| C₁₀ aromatics | 0.2 |
| C₁₁ aromatics | 0.2 |

While it will be appreciated that the light reformate analysis is dependent upon the composition of the raw naphtha, all light reformates contain some of the aryl compounds shown above to a greater or lesser extent.

One preferred embodiment is a process for a debenzening and isomerizing a full boiling range naphtha stream comprising the steps of:

(a) feeding a full boiling range C₅-C₈ naphtha stream containing benzene to a distillation column reactor into a feed zone;

(b) feeding a gas stream containing hydrogen into said feed zone; and

(c) concurrently:
(1) contacting said full boiling range naphtha stream and said gas stream with a fixed bed hydrogenation catalytic distillation structure in a distillation reaction zone under hydrogenation conditions; and
(2) fractionating said reaction mixture to:
   (i) maintain a fraction comprising benzene contained in said full boiling range naphtha stream in the distillation reaction zone sufficiently to selectively react at least a portion of said benzene with said hydrogen to form a reaction mixture containing cyclohexane, unreacted benzene and unreacted hydrogen,
   (ii) separate lighter portion containing cyclohexane and benzene from heavier components; and
   (iii) contact said cyclohexane with a selective isomerization catalytic distillation structure in a distillation reaction zone to produce methyl cyclopentane; and

(d) withdrawing a C₆ and lighter stream containing methyl cyclopentane from distillation column reactor as overheads;

(f) withdrawing a C₇ and heavier stream from the distillation column reactor as bottoms;

(g) condensing the C₅ and heavier compounds in said overheads and separating any unreacted gas from said overheads; and

(h) returning a portion of said condensed overheads to said distillation column reactor as reflux.

Hydrogenation Catalyst

The hydrogenation catalyst may comprise substantially any catalyst capable of catalyzing the hydrogenation of benzene to cyclohexane. Such a catalyst will comprise a Group VIII metal on a porous inorganic oxide support, for example Group VIII metals of the Periodic Table of Elements as the principal catalytic component, alone or with promoters and modifiers such as palladium/gold, palladium/silver, cobalt/zirconium, nickel preferably deposited on a support such as alumina, fire brick, pumice, carbon, resin, silica, an aluminosilicate, such as a zeolite or the like. The preferred Group VIII metals include platinum and palladium with platinum being more preferred. Among the metals known to catalyze the hydrogenation reaction are platinum, rhenium, cobalt, molybdenum, nickel, tungsten and palladium. Generally, commercial forms of catalyst use supported oxides of these metals. The oxide is reduced to the active form either prior to use with a reducing agent or during use by the hydrogen in the feed. Preferred hydrogenation catalysts include platinum on alumina and platinum on a zeolite with alumina binder added for strength. Suitable zeolites include faujasite, mordenite and synthetic alumino-silicates.

Isomerization Catalyst

The isomerization catalyst can comprise a Group VIII metal, preferably platinum or palladium, more preferably platinum, on a porous inorganic oxide support, for example alumina, silica/alumina or an alumino-silicate such as a zeolite. If the support itself does not have sufficient acidity to promote the needed isomerization reactions such acidity can be added. Suitable isomerization catalysts comprise platinum on chloride alumina and platinum on a zeolite which has an acidic function for promoting isomerization. Suitable zeolites include faujasite, mordenite and synthetic alumino-silicates.

Process

Distillation Column Reactor

The hydrogenation is the key step. When the hydrogenation is conducted in the catalytic distillation mode, the hydrogenation is carried out in a catalyst-packed column which can be appreciated to contain a vapor phase and some liquid phase as in any distillation. The distillation column reactor is operated at a pressure such that a reaction mixture is boiling in the bed of catalyst. The present process operates at overhead pressure of said distillation column reactor in the range between 0 and 350 psig, preferably 250 or less, preferably 35 to 120 psig and temperatures in said distillation reaction bottoms zone in the range of 100 to 500° F., preferably 150 to 400° F., e.g. 212 to 374° F. at the requisite hydrogen partial pressures. Under these conditions the benzene is maintained in the hydrogenation catalyst a sufficient time to obtain benzene conversions of over 50 wt. %, usually over 80 wt. % and at the same time remove the cyclohexane and unreacted benzene as overheads. By placing an isomerization catalyst bed in the form of a distillation structure above the hydrogenation catalyst bed the cyclohexane is isomerized to methyl cyclopentane as are the isoparaffins in this fraction.

The feed weight hourly space velocity (WHSV), which is herein understood to mean the unit weight of feed per hour entering the reaction distillation column per unit weight of catalyst in the catalytic distillation structures, may vary over a very wide range within the other condition perimeters, e.g. 0.1 to 35.

In order to maintain benzene in a light naphtha reformate within the catalyst bed, for example, the pressure can be at 75 psig to maintain an overhead temperature of about 275° F., mid reflux of about 300° F. and a bottoms temperature of about 400° F. The temperature in the catalyst bed would be around 270° F. The hydrogenation is primarily carried out with the benzene to produce a gasoline component of desirable properties (a bottoms product containing toluene), which can then be recombined with the other components of the reformate.

After hydrogenation, the stream, which in preferred embodiments includes C₅-C₆ paraffins, is contacted in an isomerization zone, for example, positioned above the hydrogenation zone in the same reaction distillation column with an isomerization catalyst under isomerization conditions to produce an isomerized product. The ranges of temperature and pressure can be a temperature which falls within those for the hydrogenation.

To provide the desired degree of temperature and residence time control, a process and apparatus are provided wherein the reaction liquid is boiling within a distillation column reactor. Overheads are withdrawn and condensed with some of the condensate being returned to the distillation column reactor as reflux. The advantage of the present process is that due to the continual reflux a portion of the selected aromatic is always condensing on the catalyst structure.

Additionally, in the catalytic distillation mode the vaporization of the liquid feed removes a substantial amount of the exothermic heat of reaction. Since the liquid is at the boiling point in the reactor, the temperature may be controlled by the pressure. An increase in pressure increases the temperature and a decrease in pressure decreases the temperature.

Part of the distillation column is preferably packed with catalytic material which incorporates the suitable catalysts discussed above. The catalysts may be combined with other suitable materials and made into a shape of conventional distillation packing such as Penn State packings, Pall rings, saddles or the like. Other packing shapes include Gempak high efficiency structured packing and Cascade MiniRings. The catalytic material may be located either in a series of zones or one particular part of the distillation column where the liquid and the vapor streams are in contact. Because the hydrogenation reactions are exothermic, dividing the catalytic material into several zones will help minimize local high temperatures. The material is arranged such that it provides a sufficient surface area for catalytic contact of the reaction streams.

In the catalytic distillation mode, the catalytic material is a component of a distillation system functioning as both a catalyst and distillation packing, i.e., a packing for a distillation column having both a distillation function and a catalytic function. The catalyst is prepared in the form of a catalytic distillation structure. More particularly the hydrogenation catalyst is generally a metal supported on an alumina carrier in the form of extrudates or spheres. The extrudates or spheres are placed in porous containers and suitably supported in the distillation column reactor to allow vapor flow through the bed, yet provide a sufficient surface area for catalytic contact.

The catalytic distillation process employs a catalyst system (See U.S. Pat. Nos. 5,730,843; 4,302,356; and 4,215,011) which provides for both reaction and distillation concurrently in the same reactor, at least in part within the catalyst system. The method involved is briefly described as one where concurrent reaction and distillation occur in combination reaction-distillation structures which are described in several U.S. patents, namely U.S. Pat. Nos. 4,242,530; 4,250,052; 4,232,177; 4,302,356; 4,307,254; and 4,336,407. Additionally U.S. Pat. Nos. 4,302,356 and 4,443,559 disclose catalyst structures which are useful as distillation structures.

The catalyst component may take several forms. In the case of particulate catalytic material, generally from 60 mm to about 1 mm down through powders, is enclosed in a porous container such as screen wire, or polymeric mesh. The material used to make the container must be inert to the reactants and conditions in the reaction system. The screen wire may be aluminum, steel, stainless steel, and the like. The polymer mesh may be nylon, Teflon, or the like. The mesh or threads per inch of the material used to make the container is such that the catalyst is retained therein and will not pass through the openings in the material. Although the catalyst particles of about 0.15 mm size or powders may be used and particles up to about ¼ inch diameter may be employed in the containers.

The mole ratio of hydrogen to benzene fed to the distillation column reactor is preferably between 2:1 and 41:1.

The hydrogenation of benzene is an exothermic reaction. In the catalytic distillation mode, because the reaction is occurring concurrently with distillation, the initial reaction products and other stream components are removed from the reaction zone as quickly as possible reducing the likelihood of side reactions. Second, in both the catalytic distillation and the downflow mode because all the components are boiling the temperature of reaction is controlled by the boiling point of the mixture at the system pressure. The heat of reaction simply creates more boil up, but no increase in temperature at a given pressure.

The hydrogen to hydrocarbon mole ratio is usually 1:1 or greater for the hydrogenation. The hydrogen stream is at an effective hydrogen partial pressure of at least about 0.1 psia to less than 70 psia, preferably less than 50 psia in the range of 2 to 25 psia.

The distillation column reactor may be operated as a splitter with the C₆ and lighter material going overhead and the C₇ and heavier going out as bottoms. In the catalytic distillation process the temperature is controlled by operating the reactor at a given pressure to allow partial vaporization of the reaction mixture. The exothermic heat of reaction is thus dissipated by the latent heat of vaporization of the mixture. The vaporized portion is taken as overheads and the condensible material condensed and returned to the column as reflux.

The downward flowing liquid causes additional condensation within the reactor as is normal in any distillation. The contact of the condensing liquid within the column provides excellent mass transfer for dissolving the hydrogen within the reaction liquid and concurrent transfer of the reaction mixture to the catalytic sites. It is thought that this condensing mode of operation results in the excellent conversion and selectivity of the instant process and allows operations at the lower hydrogen partial pressures and reactor temperatures noted. A further benefit that this reaction may gain from catalytic distillation is the washing effect that the internal reflux provides to the catalyst thereby reducing polymer build up and coking. Internal reflux may vary over the range of 0.2 to 20 L/D (wt. liquid just below the catalyst bed/wt. distillate) which gives excellent results.

A particularly unexpected benefit of the present process centers on the combined reaction distillation going on in the column. The reformate comprises a mixture of organic aromatic compounds boiling over a range. The product from the hydrogenation can be tailored by adjusting the temperature in the column to fractionate the reformate feed concurrently with the reaction of hydrogen and aromatic compound and the distillation of the hydrogenation product. Any cut can be made that is within the capacity of the equipment. For example the light end of the reformate along with the cyclohexane can be can be moved up in the distillation column reactor into a bed of isomerization catalyst and the methyl cyclopentane and light isoparaffins removed as overheads, heavies such as toluene taken as bottoms and a high concentration of benzene maintained in the portion of the column containing the hydrogenation catalytic distillation structure for a sufficient period to hydrogenate a portion of the benzene. The location of the catalyst beds can also be tailored for optimum results.

Referring now to the FIGURE there is shown a flow diagram of one embodiment of the invention. A light naphtha containing benzene is fed via line 1 and hydrogen via line 2 both being combined in line 3 which feeds the hydrogen and naphtha below the hydrogenation catalytic distillation structure 12 contained in distillation column reactor 10. Heat necessary for start up and to balance the process is provided by circulating the bottoms stream 4 through reboiler 50 and return line 5. The benzene is boiled up into the bed where it reacts with hydrogen to form a reaction mixture containing the reaction product, cyclohexane. The exothermic heat of reaction causes more boil up of the reaction mixture with the vaporized portion ascending the column into isomerization catalytic distillation structure 14 where cyclohexane is isomerized to methyl cyclopentane and C₅-C₆ normal paraffins are isomerized to the isoparaffins and exit the column as overheads via flow line 7. Unreacted hydrogen also exits with the overheads. The gaseous overheads are passed through condenser 30 where substantially all of the C₆− and cyclopentane are condensed. The overheads stream is then passed to receiver/separator 40 where the gas which is mostly hydrogen is separated and the liquid collected. The gas is removed via line 9 for recycle or use later in the process.

A portion of the condensed liquid is returned to the distillation column as reflux via flow line 8 where it provides additional cooling and condensing within the column. Overhead products are taken via flow line 6. The bottoms, containing toluene and C₇+ material, are removed via flow line 4 with a portion being recirculated through reboiler 50 and flow line 5. Both the C₇+ fraction and the C₅− fraction are preferably kept separate so that optimum blending can be obtained.

Downflow Reactor

A downflow reactor such as that described in U.S. Pat. No. 6,413,413, may be used for hydrotreating petroleum feed comprising concurrently passing a petroleum feed and hydrogen downflow through a reaction zone containing a catalyst at a pressure of less than 300 psig pressure, preferably less than 275 psig, for example less than 200 psig, and for example at least about 100 psig at a temperature within the range of 300° F. to 700° F. to produce an effluent, in which the temperature and pressure are adjusted such that the temperature of the effluent is above its boiling point and below its dew point, whereby at least a portion but less than all of the material in said reaction zone is in the vapor phase. The reaction mixture and the reaction effluent form a very complex mixture of hydrocarbons, boiling over a range of temperatures and that similarly there is a range of dew points. Thus, the actual temperature of the reaction effluent (which is very similar in composition to that of the petroleum feed but having a reduced olefin content which also occurs during the sulfur compound removal) is the temperature at a given pressure at which some lower boiling components are vaporized, but at which some of the higher boiling components are not boiling, i.e., some higher boiling components are below their dew point. Therefore, in the downflow reaction system there are always two phases.

The nature of some streams that are treated in the downflow process is such that within the process operating variables, the stream is totally vaporized and thus the benefit of the invention is not obtained. In these cases a higher boiling petroleum component is added to the stream, i.e., the “target” stream to be treated and the conditions adjusted so as to vaporize whatever portion of the target stream is necessary to reduce the total sulfur content, while the higher boiling petroleum component provides the liquid component of the reaction system.

The reactor is a fixed bed reactor, with the hydrogenation catalyst loaded into the reactor in its particulate form to fill the hydrogenation reaction zone, although there may be one or more such continuous beds, separated by spaces devoid of catalyst. The isomerization catalyst is loaded below the hydrogenation zone in one or more beds as described to form the isomerization zone. The beds are vertical with the feed passing downward through the beds and exiting after reaction through the lower end of the reactor. The reactor may be said to run in a quasi-isothermal manner.

The present process allows for the use of much lower hydrogen partial pressures and somewhat lower temperatures than normal processes.

The invention claimed is:

1. A process for a debenzening complex refinery streams comprising the steps of:
hydrogenating a complex refinery stream containing the benzene to produce cyclohexane and
isomerizing the cyclohexane to methyl cyclopentane.

2. The process according to claim 1 wherein said hydrogenating and isomerizing are carried out under conditions of concurrent reaction and distillation.

3. The process according to claim 2 wherein said hydrogenating and isomerizing are carried out under conditions of concurrent reaction and distillation in separate zones of the same distillation column reactor.

4. The process according to claim 2 wherein said hydrogenating and isomerizing are carried out under conditions of concurrent reaction and distillation in separate zones of different distillation column reactors.

5. The process according to claim 1 wherein said hydrogenating and isomerizing in separate zones are carried out under conditions of downflow reaction whereby at least a portion but less than all of the material in said reaction zones is in the vapor phase.

6. The process according to claim 5 wherein said hydrogenating and isomerizing are carried out at a pressure of less than 300 psig pressure, at a temperature within the range of 300° F. to 700° F.

7. The process according to claim 6 wherein said hydrogenating and isomerizing are carried out at a pressure of at least about 100 psig and less than 275 psig pressure, at a temperature within the range of 300° F. to 700° F.

8. A process for a debenzening and isomerizing complex refinery streams comprising: (a)feeding a complex refinery stream, containing benzene, to a distillation column reactor into a feed zone and hydrogen into said feed zone; and (b) concurrently: (1) contacting a benzene rich portion of said complex refinery stream and said hydrogen with a fixed bed selective hydrogenation catalytic distillation structure in a distillation reaction zone under hydrogenation conditions; and (2) fractionating said reaction mixture to: (i) to bring said benzene into the distillation reaction zone to selectively react at least a portion thereof with said hydrogen to form a reaction mixture containing cyclohexane and unreacted hydrogen and (ii) fractionating cyclohexane from said reaction mixture; and (3) contacting said cyclohexane and hydrogen with a selective isomerization catalytic distillation structure in a distillation reaction zone to produce methyl cyclopentane.

9. The process according to claim 8 wherein the overhead pressure of the distillation column reactor is between 10 and 120 psig.

10. The process according to claim 9 wherein the bottoms temperature of the distillation column reactor is between 212 and 450° F.

11. The process according to claim 8 wherein the hydrogen partial pressure is less than 70 psia.

12. The process according to claim 8 wherein the overhead pressure is between 0 and 350 psig.

13. The process according to claim 8 wherein hydrogen is fed below said selective isomerization catalytic distillation structure.

14. A process for a debenzening and isomerizing a full boiling range naphtha stream comprising the steps of:
(a) feeding a full boiling range C₅-C₈ naphtha stream containing benzene to a distillation column reactor into a feed zone;
(b) feeding a gas stream containing hydrogen into said feed zone; and
(c) concurrently:
(1) contacting said full boiling range naphtha stream and said gas stream with a fixed bed hydrogenation catalytic distillation structure in a distillation reaction zone under hydrogenation conditions; and
(2) fractionating said reaction mixture to:
(i) maintain a fraction comprising benzene contained in said full boiling range naphtha stream in the distillation reaction zone sufficiently to selectively react at least a portion of said benzene with said hydrogen to form a reaction mixture containing cyclohexane, unreacted benzene and unreacted hydrogen,
(ii) separating lighter portion containing cyclohexane and benzene from heavier components; and
(iii) contacting said cyclohexane with a selective isomerization catalytic distillation structure in a distillation reaction zone to produce methyl cyclopentane; and
(d) withdrawing a C₆ and lighter stream containing methyl cyclopentane from distillation column reactor as overheads;
(e) condensing the C₅ and heavier compounds in said overheads and separating any unreacted gas from said overheads; and
(f) returning a portion of said condensed overheads to said distillation column reactor as reflux.

15. The process according to claim 14 wherein the overhead pressure of the distillation column reactor is between 10 and 120 psig.

16. The process according to claim 15 wherein the bottoms temperature of the distillation column reactor is between 212 and 450° F.

17. The process according to claim 14 wherein the hydrogen partial pressure is less than 70 psia.

18. The process according to claim 17 wherein the hydrogen partial pressure is between 0.1 and 50 psi.

19. The process according to claim 18 wherein the hydrogen partial pressure is between 2 and 30 psia.

20. The process according to claim 14 wherein the overhead pressure is between 0 and 350 psig.
